(19) United States
(12) Patent Application Publication
(10) Pub. No.: US 2025/0387420 A1
(43) Pub. Date: Dec. 25, 2025

(54) Prevention of Nausea and Vomiting from Antibody-Drug Conjugates and other Long-Acting Emetogenics

(71) Applicant: Helsinn Healthcare SA, Lugano / Pazzallo (CH)

(72) Inventors: Silvia Olivari, Ticino (CH); Andrea Pontiggia, Lomazzo Como (IT)

(21) Appl. No.: 19/243,729

(22) Filed: Jun. 20, 2025

Related U.S. Application Data
(60) Provisional application No. 63/663,632, filed on Jun. 24, 2024.

(51) Int. Cl. A61K 31/675; A61K 31/473; A61K 31/496; A61K 31/5513; A61K 31/573; A61K 45/06; A61P 1/08

(52) U.S. Cl. CPC A61K 31/675; A61K 31/473; A61K 31/496; A61K 31/5513; A61K 31/573; A61K 45/06; A61P 1/08

(57) ABSTRACT

Methods of preventing the side effects of antibody drug conjugate therapies and other long acting emetogenic therapies such as nausea and vomiting, particularly in the long-delayed phase (i.e. >120 hours).

### RELATIONSHIP TO PRIOR APPLICATIONS

This application claims to priority to U.S. Provisional Application No. 63/663,632, filed Jun. 24, 2024 (expired as of Jun. 24, 2025).

### TECHNICAL FIELD

The present disclosure relates to antibody drug conjugate therapies and other long acting emetogenic therapies, and to methods of preventing side effects such as nausea and vomiting from such therapies, particularly in the long-delayed phase (i.e. >120 hours).

### BACKGROUND

Nausea and vomiting are commonly experienced with anticancer treatment and several antiemetic dosing regimens have been approved by regulatory authorities, or endorsed by clinical guidelines, to prevent nausea and vomiting for a period of five days after such cancer treatment is administered. These regimens are typically based on one or a combination of steroids, 5-HT3 receptor antagonists, NK1 receptor antagonists, and olanzapine, and vary depending on the propensity of the anticancer treatment to cause nausea and vomiting.

The five days immediately following the anticancer treatment are commonly divided into an acute phase (0-24 hours) and a delayed phase (24-120 hours), and it is generally accepted that different biological mechanisms are involved in the emetic response during these phases. However, less is known about emetogenic pathways beyond 120 hours, or how to control emesis that emerges after 120 hours. (Farhat J et al., Breast Cancer. 2025 Jan. 29; 32 (2):278-285.). The issue has received considerable attention of late, with emerging data indicating that nausea and vomiting following treatment by antibody-drug conjugates (ADCs) and standard chemotherapy regimens can occur for an even longer time than originally contemplated, referred to herein as a “long-delayed” phase, causing detrimental effects on quality of life and poor adherence to targeted treatment. Iihara H. et al, Journal of Cancer (2023) 14 (14):2644-2654; Chow R. et al, Supportive Care in Cancer (2023) 31:505.

As explained by Notini G. et al, Frontiers in Oncology (2024), pharmacokinetic studies show that free molecules of deruxtecan, the chemotherapeutic agent administered by the ADC trastuzumab deruxtecan (T-DXd), are released into the systemic circulation at levels that remain pharmacologically active for extended durations, supporting the hypothesis that circulating free payloads contribute not only to the efficacy but also the toxicity profile of T-DXd.

Some research has been undertaken to determine effective treatments during the long-delayed phase, but none has proven the effectiveness of any regimen to control nausea and vomiting during this long-delayed phase, or identified an optimal dosing regimen. Aoyama et al., for example, compared doublet (palonosetron+dexamethasone) and triplet (fosaprepitant+palonosetron+dexamethasone) antiemetic regimens for 21 days after trastuzumab deruxtecan administration, and found that the triplet regimen performed worse than the doublet regimen. (Aoyama T. et al., International Journal of Clinical Oncology (28 Apr. 2025)). Inui et al., recently performed a pooled analysis of randomized phase II and phase III studies to compare the efficacy of netupitant with aprepitant in patients receiving cisplatin-based chemotherapy, for a period of 168 hours, and concluded that netupitant had favorable comparative efficacy to aprepitant during the 168 hours following highly emetogenic cisplatin-based therapy. (Inui N. et al, Adv Ther (2023) 40:4928-4944). Notini G. et al, Frontiers in Oncology (2024) report a retrospective evaluation of cases in which ADC therapy was administered, along with either doublet (palonosetron+dexamethasone) or triplet (netupitant+palonosetron+dexamethasone) antiemetic regimens, and found no difference in nausea between the two groups, but a reduction in vomiting in the triplet group. Since the paper was merely a retrospective chart review, the paper does not report many details about the antiemetic regimen, the ADC administered or its regimen, the timing at which the antiemetic effects were measured, or effects of treatment on other chemotherapy-associated symptoms.

A simplified approach to estimate the antiemetic efficacy is the evaluation of the NK1 receptor occupancy. Bergstrom M. et al, Biol Psychiatry (2004) 55:1007-1012, for example, report that >90% NK1 receptor occupancy rates are necessary for aprepitant efficacy. Spinelli T. et al, The Journal of Clinical Pharmacology (2013) 54 (1) 97-108 report that netupitant achieves 90% receptor occupancy at plasma concentrations of 225 ng/mL, and Gilmore J and Bernareggi A, The Journal of Clinical Pharmacology 59 (4) 472-487 (2019) report that plasma concentrations fall below 100 ng/mL just 48 hours after a single 300 mg oral netupitant dose is administered.

Further complicating the evaluation of antiemetic regimens combating ADC therapy is the insurgence of ADC side effects different from nausea and vomiting, such as, for example, fatigue, loss of appetite and weight loss. All of these complications can negatively affect patient's resistance and capability to fully comply with the required chemotherapy treatment.

What is needed are antiemetic dosing regimens capable of tackling the particularly long-term off-target effects of ADC therapies and other long acting chemotherapies and immunotherapies. Ideally, these regimens would be able to prevent long-delayed nausea and vomiting during the administration of such treatments, even for those arising beyond 240 hours, which represents a largely unexplored and unaddressed treatment area, and could even be extended to other emetogenic chemotherapies, particularly highly emetogenic and moderately emetogenic chemotherapies. Regimens preventing ADCs and other treatment side-effects other than nausea and vomiting are also needed especially in the long-delayed phases of treatment. Such regimens should not only control nausea and vomiting, but also offer a higher quality of life and promote the compliance necessary to finish a complete course of the treatment without dose reduction caused by tolerability issues.

### SUMMARY OF THE DISCLOSURE

It has unexpectedly been discovered that netupitant is effective for up to 480 hours at preventing nausea and vomiting and other side effects from ADC and other long-acting emetogenic therapies, including chemotherapies and monoclonal antibody therapies. Particularly significant is the presence of effects also in the very late stages, generally between >240 hours and up to 480 hours after administration of the chemotherapeutic agent, a time window so far unexplored for antiemetic treatments representing an unmet need area in which significant ADC-caused emesis and related side effects are present. Applicant's studies have for the first time revealed for netupitant a kinetics of receptor occupancy significantly different from that of the golden standard for NK1 antagonists, aprepitant, also studied comparatively for the first time by the inventors: these comparative studies have supported a much longer-lasting activity of netupitant, going beyond a mere reflection of differences in plasmatic half-life among the two drugs.

Thus, in one embodiment the disclosure provides a method of preventing ADC-induced nausea and/or vomiting, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

In another embodiment the disclosure provides a method of achieving a positive no significant nausea outcome following ADC administration, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

In another embodiment the disclosure provides a method of preventing an ADC-induced side effect selected from fatigue, loss of appetite, and weight loss, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy. A preferred ADC-induced side effect treated by the present methods is fatigue: the risk of developing this side effect is high among ADC-treated patients, especially for those experiencing early-onset emesis, arising during the first ADC treatment cycle; the present treatment has shown a significant antiemetic efficacy already at this early stage, resulting in a correspondingly strong reduction of the risk of developing fatigue.

In another embodiment the disclosure provides a method of preventing ADC-, HEC-, or MEC-induced nausea and/or vomiting, during a long-delayed phase, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

In another embodiment the disclosure provides a method of achieving a positive no significant nausea outcome following ADC, HEC, or MEC administration, during a long-delayed phase, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

In another embodiment the disclosure provides a method of preventing an ADC-induced side effect selected from fatigue, loss of appetite, and weight loss, during a long-delayed phase, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy; preferably, the ADC-induced side effect is fatigue.

In another embodiment the disclosure provides a method of preventing ADC-induced nausea and/or vomiting, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

In another embodiment the disclosure provides a method of achieving a positive no significant nausea outcome following ADC administration, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

In another embodiment the disclosure provides a method of preventing an ADC-induced side effect selected from fatigue, loss of appetite, and weight loss, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy; preferably, the ADC-induced side effect is fatigue.

In another embodiment the disclosure provides a method of preventing ADC-, HEC-, or MEC-induced nausea and/or vomiting in a human subject in need thereof, during a long-delayed phase, to a significantly greater extent than an aprepitant or fosaprepitant regimen, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

In another embodiment the disclosure provides a method of achieving a positive no significant nausea outcome following ADC, HEC, or MEC administration, during a long-delayed phase, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

In another embodiment the disclosure provides a method of preventing an ADC-, HEC-, or MEC-induced side effect selected from fatigue, loss of appetite, and weight loss, during a long-delayed phase, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy; preferably, the ADC-induced side effect is fatigue.

These and other aspects of embodiments of the disclosure will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE FIGURES

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements and have been solely selected for ease of recognition in the figures.

FIG. 1 plots predicted NK1 receptor occupancy rates following administration of standard doses of netupitant and aprepitant, for up to 480 hours, using the model described in Baron-Hay S et al, Supp Care Cancer (2019) 27 (4): 1309-1317.

### DETAILED DESCRIPTION

In the following description, certain specific details are set forth in order to provide a thorough understanding of various embodiments of the disclosure. However, one skilled in the art will understand that embodiments of the disclosure may be practiced without these details.

### Definitions and Use of Terms

Unless the context requires otherwise, throughout the present specification and claims, the word “comprise” and variations thereof, such as “comprises” and “comprising,” are to be construed in an open, inclusive sense (i.e., as “including, but not limited to”).

Reference throughout this specification to “one embodiment” or “an embodiment” or “some embodiments” means that a particular feature, structure or characteristic described in connection with the embodiment is included in at least one embodiment of the present disclosure. Thus, the appearances of the phrases “in one embodiment” or “in an embodiment” or “in some embodiments” in various places throughout this specification are not necessarily all referring to the same embodiment. Furthermore, the particular features or characteristics may be combined in any suitable manner in one or more embodiments.

Any numerical value recited herein can be modified by the term “about” to compensate for the inherent variability in precise numerical values. To the extent the term is deemed vague or indefinite, “about” can be substituted with ±10% of the given value. In addition, when the term “about” is used herein, it may also be substituted with ±5% of the given value to create additional embodiments, or ±2% of the given value to create even further embodiments.

In various embodiments herein, a treatment effect such as a reduction in the severity or duration of a symptom is compared to the effect observed in the subject in the absence of such treatment. When such a comparison is made, it will be understood that the treatment effect can be identified, evaluated or quantified based on a comparison to a placebo or historical control, or potentially an active control. In any of these embodiments, the treatment effect is preferably statistically significant (p<0.05) and clinically meaningful.

The methods described herein have as their object various therapeutic effects, described variously as the “prevention of nausea and/or vomiting,” the “achieving no significant nausea,” prevention of side effects,” “attaining complete response” and the like. Whenever a treatment object is disclosed herein, it will be understood that the antiemetic regimen administered is therapeutically effective to accomplish the object, and actually achieves the therapeutic effect, preferably to a degree which is both statistically significant (p<0.05) and clinically meaningful. Conversely, whenever a method is said to achieve a particular therapeutic effect, such as nausea prevention or complete response, or a particular treatment effect during a long-delayed phase, or a particular treatment effect relative to a comparator regimen, it will be understood that the particular treatment effect is an object of the recited method.

When a “phase” is expressed as a time period herein, it will be understood to be measured from the time point beginning immediately after the administration of an emetogenic therapy is initiated, also referred to herein as to. When a range for a time period is expressed herein, the range is measured from the beginning of the first time point of the range to the end of the second time point of the range. Thus, a range of 0-2 hours, corresponds to the time period extending from t₀ to t₁₂₀ₘ. The time period can be expressed in hours or days, and the units of measure are interchangeable. Thus, the time period spanning hours 0-120 is the same as the time period spanning days 0-5, and the time period spanning hours 0-240 is the same as the time period spanning of days 1-10.

The “acute phase” refers to the time-period spanning hours 0-24 after the administration of an antibody drug conjugate, HEC, or MEC, or other emetogenic therapy.

The “delayed phase” refers to the time-period spanning hours 24-120 after the administration of an antibody drug conjugate, HEC, or MEC, or other emetogenic therapy.

The “overall phase” refers to the time-period spanning hours 0-120 after the administration of an antibody drug conjugate, HEC, or MEC, or other emetogenic therapy.

The “long-delayed phase” refers to a time interval starting at a time point no sooner than 120 hours and preferably ending at a time point no later than 480 hours, after the administration of an antibody drug conjugate, HEC, or MEC, or other emetogenic therapy. Exemplary “long-delayed phases” thus include any time-periods spanning hours from 120 to 480 hours after the administration of an antibody drug conjugate, HEC, or MEC, or other emetogenic therapy; among them, a first exemplary group of long delayed phases includes the time periods 120-168, 120-216, 120-240, 120-480 hours after the administration of an antibody drug conjugate, HEC, or MEC, or other emetogenic therapy; a second exemplary group of long delayed phases includes the time periods 120-264, 120-312, 120-360, 120-408, 120-456, 120-480 168-216, 168-264, 168-312, 169-360, 168-408, 168-456, 168-480, 216-264, 216-312, 216-360, 216-408, 216-456, 216-480 hours after the administration of an antibody drug conjugate, HEC, or MEC, or other emetogenic therapy; a third exemplary group of long delayed phases includes the time periods >240-360, >240-480, 264-312, 264-360, 264-408, 264-456, or 264-480, 312-360, 312-408, 312-456, or 312-480, 360-408, 360-456, 360-480, 408-456, 408-480, 456-480 hours after the administration of an antibody drug conjugate, HEC, or MEC, or other emetogenic therapy; a fourth exemplary group of long delayed phases includes all the previous three groups.

Whenever a therapeutic effect is said to occur during the “long-delayed phase” it will be understood that it can occur during any of the foregoing time-periods.

The “long-overall phase” refers to a time interval starting at hour zero and ending at a time point later than 120 hours, but no later than 480 hours, after the administration of an antibody drug conjugate, HEC, or MEC, or other emetogenic therapy. Exemplary “long-overall phases” thus include the time-periods spanning hours 0-168, 0-216, 0-240, 0-264, 0-312, 0-360, 0-408, 0-456, or 0-480 after the administration of an antibody drug conjugate, HEC, or MEC, or other emetogenic therapy. Whenever a therapeutic effect is said to occur during the “long-overall phase” it will be understood that it can occur during any of the foregoing time-periods.

“ADC-induced nausea and/or vomiting” refers to nausea and/or vomiting induced by the administration of an antibody drug conjugate. When a treatment is said to prevent ADC-induced nausea and/or vomiting, it will be understood to prevent ADC-induced nausea, ADC-induced vomiting, or both.

An “emetogenic therapy” refers to any of the various treatments described herein, including ADC therapy, chemotherapy, and monoclonal antibody therapy, including both highly emetogenic therapy and moderately emetogenic therapy.

An “antibody drug conjugate” or “ADC” refers to a substance made up of a monoclonal antibody chemically linked to a drug. The monoclonal antibody binds to specific proteins or receptors found on certain types of cells, including cancer cells. The linked drug enters these cells and kills them without harming other cells. Exemplary antibody drug conjugates approved by the United States Food and Drug Agency include gemtuzumab ozogamicin (Mylotarg®), brentuximab vedotin (Adcetris®), trastuzumab emtansine (Kadcyla®), inotuzumab ozogamicin (Besponsa®), polatuzumab vedotin (Polivy®), enfortumab vedotin (Padcev®), trastuzumab deruxtecan (Enhertu®), sacituzumab govitecan (Trodelvy®), loncastuximab tesirine (Zynlonta®), tisotumab vedotin (Tivdak®), mirvetuximab soravtansine (Elahere®). Exemplary antibody drug conjugates unapproved or recently approved by the United States Food and Drug Agency (as of Jun. 21, 2024) include datopotamab deruxtecan, luveltamab tazevibulin, patritumab deruxtecan, mecbotamab vedotin, sacituzumab tirumotecan, telisotuzumab vedotin, trastuzumab auristatin, trastuzumab rezetecan, and zilovertamab vedotin. Preferred ADC used in the invention are selected from trastuzumab deruxtecan, sacituzumab govitecan and datopotamab deruxtecan. Preferred ADC used in the invention are selected from trastuzumab deruxtecan, sacituzumab govitecan and datopotamab deruxtecan.

Whenever a regimen is described herein based on ADC administration, it will be understood also that the regimen can be based on therapeutic antibodies. Antibodies are immune system proteins that can be created in vivo or ex vivo through various laboratory techniques including recombinant chemistry. Many antibodies are used to treat cancer and fall within the scope of the current disclosure. They are a type of targeted cancer therapy, which means they are designed to interact with specific targets. Some antibodies are also immunotherapy because they help turn the immune system against cancer. For example, some monoclonal antibodies mark cancer cells so that the immune system will better recognize and destroy them. An example is rituximab, which binds to a protein called CD20 on B cells and some types of cancer cells, causing the immune system to kill them. Other monoclonal antibodies bring T cells close to cancer cells, helping the immune cells kill the cancer cells. An example is blinatumomab, which binds to both CD19, a protein found on the surface of leukemia cells, and CD3, a protein on the surface of T cells. This process helps the T cells get close enough to the leukemia cells to respond to and kill them.

Monoclonal antibodies suitable for use in the present disclosure thus include, without limitation, depemokimab, apitegromab, telisotuzumab vedotin, clesrovimab, sipavibart, nipocalimab, bentracimab, datopotamab deruxtecan, zenocutuzumab, nemolizumab, zanidatamab, linvoseltamab, axatilimab, patritumab deruxtecan, tarlatamab, marstacimab, garadacimab, vilobelimab, zolbetuximab, odronextamab, crovalimab, camrelizumab, serplulimab, sugemalimab, concizumab, cosibelimab, trastuzumab duocarmazine, donanemab, narsoplimab, pozelimab, elranatamab, rozanolixizumab, talquetamab, epcoritamab, lebrikizumab, glofitamab, mirikizumab, tislelizumab, toripalimab, retifanlimab, lecanemab, teplizumab, ublituximab, mirvetuximab soravtansine, nirsevimab, tremelimumab, spesolimab, teclistamab, mosunetuzumab, tixagevimab, cilgavimab, relatlimab, tebentafusp, faricimab, sutimlimab, sotrovimab, regdanvimab, casirivimab+imdevimab, tezepelumab, tisotumab vedotin, amivantamab, anifrolumab, loncastuximab tesirine, bimekizumab, tralokinumab, evinacumab, aducanumab, dostarlimab, ansuvimab, margetuximab, naxitamab, atoltivimab, maftivimab, and odesivimab-ebgn, belantamab mafodotin, tafasitamab, satralizumab, inebilizumab, sacituzumab govitecan, teprotumumab, isatuximab, eptinezumab, [fam]-trastuzumab deruxtecan, enfortumab vedotin, crizanlizumab, brolucizumab, polatuzumab vedotin, risankizumab, romosozumab, caplacizumab, ravulizumab, emapalumab, cemiplimab, fremanezumab, moxetumomab pasudotox, galcanezumab, lanadelumab, mogamulizumab, erenumab, tildrakizumab, ibalizumab, burosumab, durvalumab, emicizumab, benralizumab, ocrelizumab, guselkumab, inotuzumab, ozogamicin, sarilumab, dupilumab, avelumab, brodalumab, atezolizumab, bezlotoxumab, olaratumab, reslizumab, obiltoxaximab, ixekizumab, daratumumab, elotuzumab, necitumumab, idarucizumab, alirocumab, mepolizumab, evolocumab, dinutuximab, secukinumab, nivolumab, blinatumomab, pembrolizumab, ramucirumab, vedolizumab, siltuximab, obinutuzumab, ado-trastuzumab emtansine, raxibacumab, pertuzumab, brentuximab vedotin, belimumab, ipilimumab, denosumab, tocilizumab, ofatumumab, canakinumab, golimumab, ustekinumab, certolizumab pegol, catumaxomab, eculizumab, ranibizumab, panitumumab, natalizumab, bevacizumab, cetuximab, efalizumab, omalizumab, tositumomab-I131, ibritumomab tiuxetan, adalimumab, alemtuzumab, gemtuzumab, ozogamicin, trastuzumab, infliximab, palivizumab, basiliximab, daclizumab, rituximab, abciximab, edrecolomab, nebacumab, and muromonab-CD3.

“Prodrug”, when referred herein to a specific drug molecule, identifies a compound chemically different from the said drug molecule which, after administration to a human or animal subject, is metabolically converted to said drug molecule. A preferred prodrug of netupitant in the present invention is fosnetupitant.

“Chemotherapy” means the treatment of disease by the use of chemical substances, especially the treatment of cancer by cytotoxic and other drugs. For purposes of this disclosure, chemotherapy does not include ADC therapy or monoclonal antibody therapy.

“Highly emetogenic chemotherapy” or “HEC” refers to chemotherapy, other than ADC therapy or monoclonal antibody therapy, in which the incidence of vomiting in the absence of antiemetic prophylaxis is >90%. See Herrstedt J. et al, ESMO Open (2024) Vol. 9 Issue 2.

In like manner, “highly emetogenic ADC or monoclonal antibody therapy” refers to ADC or monoclonal antibody therapy in which the incidence of vomiting in the absence of antiemetic prophylaxis is >90%. “Highly emetogenic long-chemotherapy” or “highly emetogenic long-ADC therapy” or “highly emetogenic long monoclonal antibody therapy” refers to highly emetogenic chemotherapy or highly emetogenic ADC therapy or highly emetogenic monoclonal antibody therapy in which the risk of nausea and vomiting during a long-delayed phase remains clinically meaningful.

Representative intravenous HEC agents thus include anthracycline/cyclophosphamide combination, carmustine, chlormethine (mechlorethamine), cisplatin, cyclophosphamide 1500 mg/m², dacarbazine, and streptozocin. See Herrstedt J. et al, ESMO Open (2024) Vol. 9 Issue 2.

“Moderately emetogenic chemotherapy” or “MEC” refers to chemotherapy in which the incidence of vomiting in the absence of antiemetic prophylaxis is 30-90%. See Herrstedt J. et al, ESMO Open (2024) Vol. 9 Issue 2. In like manner, “moderately emetogenic ADC or monoclonal antibody therapy” refers to ADC or monoclonal antibody therapy in which the incidence of vomiting in the absence of antiemetic prophylaxis is 30-90%. “Moderately emetogenic long-chemotherapy” or “moderately emetogenic long-ADC therapy” or “moderately emetogenic long-monoclonal antibody therapy” refers to “moderately emetogenic chemotherapy” or “moderately emetogenic ADC therapy” or “moderately emetogenic monoclonal antibody therapy” in which the risk of nausea and vomiting during a long-delayed phase remains clinically meaningful.

Representative MEC intravenous agents include alemtuzumab, arsenic trioxide, azacitidine, bendamustine, busulfan, carboplatin, clofarabine, cyclophosphamide <1500 mg/m², cytarabine >1000 mg/m², cytarabine/daunorubicin liposomal, daunorubicin, dinutuximab beta, doxorubicin, epirubicin, idarubicin, ifosfamide, irinotecan, irinotecan peg-liposomal, lurbinectedin, naxitamab, oxaliplatin, romidepsin, temozolomide, thiotepa, and trabectedin. See Herrstedt J. et al, ESMO Open (2024) Vol. 9 Issue 2. Herrstedt also classifies sacituzumab-govitecan and trastuzumab-deruxtecan as MEC. However, for purposes of this disclosure, antibody drug conjugates/ADC and monoclonal antibody therapies are not classified as MEC or HEC, but instead treated as distinct therapeutic modalities.

Representative HEC/MEC oral agents include abemaciclib, adagrasib, avapritinib, bosutinib, cabozantinib, ceritinib, crizotinib, cyclophosphamide, enasidenib, fedratinib, hexamethylmelamine (altretamine), lenvatinib, lomustine, midostaurin, mobocertinib, nirapari, olaparib, procarbazine, ribociclib, rucaparib, selinexor, temozolomide, imatinib, and vinorelbine. See Herrstedt J. et al, ESMO Open (2024) Vol. 9 Issue 2.

“HEC- or MEC-induced nausea and/or vomiting” refers to nausea and/or vomiting induced by the administration of HEC or MEC. When a treatment is said to prevent HEC- or MEC-induced nausea and/or vomiting, it will be understood to prevent HEC- or MEC-induced nausea, HEC- or MEC-induced vomiting, or both.

In the present methods there is no limitation as to the tumor target of the ADC or monoclonal antibody: examples of ADC-treatable and monoclonal antibody-treatable tumors are: epithelial tumors such as adenocarcinoma, squamous cell carcinoma, basal cell carcinoma, transitional cell carcinoma; mesenchymal tumors such as sarcoma; hematopoietic tumors such as leukemia, lymphoma, myeloma; neuroectodermal tumors such as glioma, neuroblastoma, medulloblastoma; germ cell tumors such as seminoma, dysgerminoma, embryonal carcinoma, choriocarcinoma; blastomas, such as neuroblastoma, retinoblastoma, nephroblastoma (Wilms tumor), hepatoblastoma, medulloblastoma: Treatable tumors can also be categorized according to the affected organ, for example brain carcinoma, head and neck carcinoma, pharynx carcinoma, larynx carcinoma, thyroid carcinoma, salivary gland carcinoma, nasopharyngeal carcinoma, lung carcinoma, mesothelioma, breast carcinoma, ductal carcinoma, lobular carcinoma, esophagus carcinoma, stomach carcinoma, colon carcinoma, rectum carcinoma, pancreas carcinoma, liver carcinoma, prostate carcinoma, kidney carcinoma, bladder carcinoma, testicular carcinoma, ovarian carcinoma, etc. A preferred tumor target for the present invention is breast carcinoma, in particular early and metastatic breast carcinoma; further preferred tumor targets are HER2 positive tumors, in particular HER2 positive breast, lung or gastric cancer, HER2 low and ultra-low breast cancer; the treatment of pan-tumors is also preferred. In all these cases, the correspondingly used ADC or monoclonal antibody can be chosen as exemplified above, and is preferably selected from trastuxumab deruxtecan, datopotamab deruxtecan or sacituzumab govitecan.

The term “aprepitant or fosaprepitant regimen” encompasses any oral or intravenous regimen of aprepitant or fosaprepitant (e.g. Emend® or Cinvanti®) of comparable antiemetic prophylactic activity to a netupitant or fosnetupitant regimen described herein, ideally as approved by the United States Food and Drug Administration (as of Jun. 21, 2024). Exemplary aprepitant and fosaprepitant regimens thus include:
Aprepitant 125 mg orally 1 hour prior to chemotherapy or ADC treatment (Day 1) and aprepitant 80 mg orally once daily in the morning on Days 2 and 3, in addition to a corticosteroid and a 5-HT3 antagonist;
Fosaprepitant dimeglumine for injection 150 mg completed approximately 30 minutes prior to chemotherapy or ADC treatment (Day 1) and aprepitant 80 mg orally once daily in the morning on Days 2 and 3, in addition to a corticosteroid and a 5-HT3 antagonist;
Fosaprepitant dimeglumine for injection 150 mg completed approximately 30 minutes prior to chemotherapy or ADC treatment (Day 1), in addition to a corticosteroid and a 5-HT3 antagonist;
130 mg aprepitant as injectable emulsion completed approximately 30 minutes prior to chemotherapy or ADC treatment (Day 1); and
100 mg aprepitant as injectable emulsion completed approximately 30 minutes prior to chemotherapy or ADC treatment (Day 1) and aprepitant 80 mg orally once daily on Days 2 and 3.

The administration of corticosteroids is a usual part of antiemetic prophylaxis: accordingly, corticosteroids may be administered, in accordance with standard knowledge, in addition to or in combination with the disclosed regimens. A preferred corticosteroid is dexamethasone; it can be administered by reference to the regimens currently approved by the United States Food and Drug Administration (as of Jun. 21, 2024), supplemented as necessary by MASCC/ESMO treatment guidelines published by Herrstedt J. et al, ESMO Open (2024) Vol. 9 Issue 2. If added to the present regimens, corticosteroids are suitably administered as independent dosage forms, i.e. they are not co-formulated with nor chemically linked to any of the drugs object of the present regimens. Using olanzapine instead of corticosteroid or with the corticosteroid is also possible.

Whereas the concomitant use of corticosteroids is permitted under the present invention, the treatments described herein permit to reduce the dose and frequency of corticosteroids, advantageously reducing the risk of corresponding undesirable side effects.

A possible interplay of corticosteroid (CS)/olanzapine (OLZ) administration with the regimens of the invention is exemplified as follows:
CS on day 1 only
Reduced dose of CS day 1, 2 and 3 or reduced dose on day 2 and 3
Reduced dose of CS day 1, 2 and 3+OLZ or reduced dose 2 and 3+OLZ
OLZ and no CS.

The netupitant or fosnetupitant antiemetic regimens disclosed herein are often described as achieving a result to a significantly greater extent than an aprepitant or fosaprepitant regimen. This means that the netupitant or fosnetupitant regimen cited achieves a result to a significantly greater extent than an aprepitant or fosaprepitant regimen of comparable prophylactic activity, as described in MASCC/ESMO antiemetic guidelines published by Herrstedt J. et al, ESMO Open (2024) Vol. 9 Issue 2 (a “comparable aprepitant or fosaprepitant regimen”). This also means that the superiority has been demonstrated in a well-controlled clinical study to a statistically significant degree (i.e. p<0.05), and ideally that the degree of superiority to an aprepitant or fosaprepitant regimen is clinically meaningful, as evaluated under existing draft and final guidance documents (as of Jun. 21, 2024) issued by the United States Food and Drug Administration, supplemented as necessary by MASCC/ESMO antiemetic guidelines published by Herrstedt J. et al, ESMO Open (2024) Vol. 9 Issue 2. Thus, an improvement of ≥10% versus a comparable aprepitant or fosaprepitant regimen would be considered clinically meaningful.

“Pharmaceutically acceptable salts” refers to any non-toxic anion or cation addition salt which addresses any of the physicochemical and/or biological issues associated with a particular drug, including aqueous solubility, stability, toxicity, absorption, and issues related to synthesis and manufacturing. A discussion of such issues is given in various formulation handbooks and guides, including, for example, Gupta D, et al, Molecules (2018) 23, 1719. Salt anions approved by the United States Food and Drug Administration, which are generally considered non-toxic, include acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydrochloride, hydrobromide, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, and tosylate. Salt cations approved by the United States Food and Drug Administration include, without limitation, aluminum, arginine, benzathine, calcium, choline, diethanolamine, ethanolamine, ethylenediamine, lysine, magnesium, histidine, lithium, meglumine, potassium, procaine, sodium, triethylamine, and zinc. When a “salt” is administered or embodied in this disclosure, it will be understood to refer to a pharmaceutically active salt.

In any of the embodiments of the current disclosure, the netupitant is preferably administered as a free base, the fosnetupitant is preferably administered as the chloride hydrochloride salt, and the palonosetron is preferably administered as the hydrochloride salt.

“CINV” means “chemotherapy induced nausea and vomiting,” and is an indication used by regulatory authorities when approving antiemetic agents for the prevention of nausea and/or vomiting following the administration of emetogenic cancer therapy.

The term “vomiting” includes both retching and retrograde expulsion of gastric contents from the body. Vomiting is synonymous with an emetic episode, as that term is used herein.

“Complete response” or “CR” rate is defined as no emetic episodes and no rescue medication use, and can be evaluated during the acute, delayed, long-delayed (in any of its permutations), overall, and/or long-overall (in any of its permutations) phases.

“Complete control” or “CC” rate is defined as no emetic episodes, no rescue medication use, and no significant nausea, and can be evaluated during the acute, delayed, long-delayed (in any of its permutations), overall, and/or long-overall (in any of its permutations) phases.

“Total control” or “TC” rate is defined as no emetic episodes, no rescue medication use, and no nausea, and can be evaluated during the acute, delayed, long-delayed (in any of its permutations), overall, and/or long-overall (in any of its permutations) phases.

“Nausea” is defined as a feeling of sickness with an inclination to vomit, and can be evaluated during the acute, delayed, long-delayed (in any of its permutations), overall, and/or long-overall (in any of its permutations) phases.

“Significant nausea” is defined as any rating greater than “mild nausea” on a four-point scale gauging none, mild, moderate, and severe nausea, and can be evaluated during the acute, delayed, long-delayed (in any of its permutations), overall, and/or long-overall (in any of its permutations) phases. Conversely, “no significant nausea” is achieved when a patient experiences no nausea or only mild nausea, and can be evaluated during the acute, delayed, long-delayed (in any of its permutations), overall, and/or long-overall (in any of its permutations) phases.

“Time to treatment failure” or “TTF” is defined as the time from the administration of an antibody drug conjugate, HEC, or MEC, or other emetogenic therapy, to the first episode of vomiting or to rescue medication use, whichever happens first.

“Prevention” of nausea and/or vomiting (including ADC-induced nausea and/or vomiting and HEC- or MEC-induced nausea and/or vomiting) refers to the prevention of any measure of nausea and/or vomiting, including nausea and vomiting, nausea (of any degree), significant nausea, severe nausea, moderate nausea, mild nausea, vomiting, the achievement of a no significant nausea outcome, the attainment of CC, the attainment of CR, the attainment of TC, or an increase in TTF. Thus, whenever a method is said to prevent nausea and/or vomiting, it will be understood that the method can also be expressed as a method of preventing nausea and vomiting, a method of preventing nausea (of any degree), a method of preventing significant nausea, a method of preventing severe nausea, a method of preventing moderate nausea, a method of preventing mild nausea, a method of preventing vomiting, a method of achieving a positive no significant nausea outcome, a method of attaining CC, a method of attaining CR, a method of attaining TC, or a method of attaining an increase in TTF.

Prevention of nausea and/or vomiting can be evaluated during the acute, delayed, long-delayed (in any of its permutations), overall, and long-overall phases (in any of its permutations). Thus, whenever a method is said to prevent nausea and/or vomiting, or any embodiment of nausea and/or vomiting, it will be understood that the method prevents nausea and/or vomiting, or any embodiment of nausea and/or vomiting, during the acute, delayed, long-delayed (in any of its permutations), overall, and long-overall phases (in any of its permutations).

### Discussion

In one embodiment the disclosure provides a method of preventing ADC-induced or antibody-induced nausea and/or vomiting, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or monoclonal antibody therapy (preferably moderately or highly emetogenic).

In another embodiment the disclosure provides a method of achieving a positive no significant nausea outcome following ADC or antibody administration, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC or monoclonal antibody therapy (preferably moderately or highly emetogenic).

In another embodiment the disclosure provides a method of preventing an ADC-induced or antibody-induced side effect selected from fatigue, loss of appetite, and weight loss, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or monoclonal antibody therapy (preferably moderately or highly emetogenic); preferably, the ADC-induced or antibody-induced side effect is fatigue.

In another embodiment the disclosure provides a method of preventing ADC-, HEC-, or MEC-induced nausea and/or vomiting, or nausea and/or vomiting induced by another emetogenic therapy, during a long-delayed phase, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or chemotherapy or monoclonal antibody therapy (preferably moderately or highly emetogenic).

In another embodiment the disclosure provides a method of achieving a positive no significant nausea outcome following ADC, HEC, or MEC administration, or administration of another emetogenic therapy, during a long-delayed phase, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or chemotherapy or monoclonal antibody therapy (preferably) moderately or highly emetogenic.

In another embodiment the disclosure provides a method of preventing an ADC-, chemotherapy- (e.g. HEC-, or MEC-) or antibody induced side effect selected from fatigue, loss of appetite, and weight loss, during a long-delayed phase, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or chemotherapy or monoclonal antibody therapy (preferably moderately or highly emetogenic); preferably, the side effect is fatigue.

In another embodiment the disclosure provides a method of preventing ADC- or antibody-induced nausea and/or vomiting, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or antibody therapy (preferably moderately or highly emetogenic).

In another embodiment the disclosure provides a method of achieving a positive no significant nausea outcome following ADC or antibody administration, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or monoclonal antibody therapy (preferably moderately or highly emetogenic).

In another embodiment the disclosure provides a method of preventing an ADC- or antibody-induced side effect selected from fatigue, loss of appetite, and weight loss, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or monoclonal antibody therapy (preferably moderately or highly emetogenic); preferably, the side effect is fatigue.

In another embodiment the disclosure provides a method of preventing ADC-, chemotherapy- (e.g. HEC-, or MEC-), or antibody-induced nausea and/or vomiting in a human subject in need thereof, during a long-delayed phase, to a significantly greater extent than an aprepitant or fosaprepitant regimen, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or chemotherapy or monoclonal antibody therapy (preferably moderately or highly emetogenic).

In another embodiment the disclosure provides a method of achieving a positive no significant nausea outcome following ADC, chemotherapy (e.g. HEC, or MEC) or antibody administration, during a long-delayed phase, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or chemotherapy or monoclonal antibody therapy (preferably moderately or highly emetogenic).

In another embodiment the disclosure provides a method of preventing an ADC-, chemotherapy- (e.g. HEC-, or MEC-) and antibody-induced side effect selected from fatigue, loss of appetite, and weight loss, during a long-delayed phase, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of ADC therapy or chemotherapy or monoclonal antibody therapy (preferably moderately or highly emetogenic); preferably, the side effect is fatigue.

In one embodiment the method prevents nausea. In one embodiment the method prevents vomiting. In one embodiment the method prevents nausea and vomiting. In one embodiment the method induces no significant nausea. In one embodiment the method prevents fatigue. In one embodiment the method prevents loss of appetite, in another embodiment the method prevents weight loss. Put another way, the method can be said to treat nausea, vomiting, significant nausea, fatigue, loss of appetite, or weight loss; preferably, the method prevents one or more of nausea, vomiting, significant nausea, and one or more of fatigue, loss of appetite, or weight loss, wherein the prevention of fatigue is preferred.

In one embodiment the antiemetic regimen comprises netupitant or a prodrug thereof (e.g. fosnetupitant), or a pharmaceutically acceptable salt of netupitant or the prodrug. In another embodiment the antiemetic regimen comprises palonosetron or a pharmaceutically acceptable salt thereof. In another embodiment the antiemetic regimen comprises a combination of (i) netupitant or a prodrug thereof (e.g. fosnetupitant), or a pharmaceutically acceptable salt of netupitant or the prodrug, and (ii) palonosetron or a pharmaceutically acceptable salt thereof.

In one embodiment the antiemetic regimen comprises a single administration of a combination of (i) netupitant or a prodrug thereof (e.g. fosnetupitant), or a pharmaceutically acceptable salt of netupitant or the prodrug, and (ii) palonosetron or a pharmaceutically acceptable salt thereof. I.e., the netupitant moiety (e.g. netupitant, fosnetupitant, or a salt thereof) and the palonosetron moiety (i.e. palonosetron or a salt thereof) are each only administered once when the antiemetic regimen is administered.

In one embodiment, the method achieves the result during a long-delayed phase. I.e., the method prevents nausea and/or vomiting, the method prevents nausea, the method prevents vomiting, the method prevents nausea and vomiting, the method induces no significant nausea, or the method prevents a side effect selected from fatigue, appetite and weight loss, during a delayed phase, i.e. during any time interval between hours 120 and 480. E.g., the method prevents nausea, the method prevents vomiting, the method prevents nausea and vomiting, the method induces no significant nausea, or the method prevents a side effect selected from fatigue, appetite and weight loss, during a time period selected from hours 120-168, 120-216, 120-264, 120-312, 120-360, 120-408, 120-456, 120-480, 168-216, 168-264, 168-312, 169-360, 168-408, 168-456, 168-480, 216-264, 216-312, 216-360, 216-408, 216-456, 216-480, 264-312, 264-360, 264-408, 264-456, or 264-480, 312-360, 312-408, 312-456, or 312-480, 360-408, 360-456, 360-480, 408-456, 408-480, 456-480, 120-240, 120-360, 120-480, 240-360, 240-480, and 360-480, preferably 120-480 and 120-240 after the administration of an antibody drug conjugate, HEC, or MEC, or other emetogenic therapy. Preferably, the method prevents nausea, vomiting, induces no significant nausea, and further prevents fatigue, during a delayed phase, i.e. during any time interval between hours 120 and 480, particularly in the time interval comprised between >240 hours and ≤480 hours.

In one embodiment, the method achieves the result to a significantly greater extent than an aprepitant or fosaprepitant regimen. I.e., the method prevents nausea and/or vomiting, the method prevents nausea, the method prevents vomiting, the method prevents nausea and vomiting, the method induces no significant nausea, the method achieves a positive no significant nausea outcome, or the method prevents a side effect selected from fatigue, appetite and weight loss, to a significantly greater extent than an aprepitant or fosaprepitant regimen. In one embodiment the method prevents nausea, the method prevents vomiting, the method prevents nausea and vomiting, the method induces no significant nausea, the method achieves a positive no significant nausea outcome, or the method prevents a side effect selected from fatigue, appetite and weight loss, during a time period selected from hours 120-168, 120-216, 120-264, 120-312, 120-360, 120-408, 120-456, 120-480, 168-216, 168-264, 168-312, 169-360, 168-408, 168-456, 168-480, 216-264, 216-312, 216-360, 216-408, 216-456, 216-480, 264-312, 264-360, 264-408, 264-456, or 264-480, 312-360, 312-408, 312-456, or 312-480, 360-408, 360-456, 360-480, 408-456, 408-480, 456-480, 120-240, 120-360, 120-480, 240-360, 240-480, and 360-480, after the administration of an antibody drug conjugate, HEC, or MEC, or other emetogenic therapy, to a significantly greater extent than an aprepitant or fosaprepitant regimen. Preferably, the method prevents nausea, vomiting, induces no significant nausea, and further prevents fatigue, during a delayed phase, i.e. during any time interval between hours 120 and 480, particularly in the time interval comprised between >240 hours and ≤480 hours.

In some embodiments, the nausea and/or vomiting comprises nausea and/or vomiting through day 7, day 10, day 15, or day 20 after the administration of the emetogenic therapy. In other embodiments, the nausea and/or vomiting comprises nausea and/or vomiting during days 6-7, 8-10, 6-10, 11-15, or 16-20, or a combination thereof, after the emetogenic therapy. In other embodiments the nausea and/or vomiting comprises nausea and/or vomiting experienced on day 6, day 7, day 8, day 9, day 10, day 11, day 12, day 13, day 14, day 15, day 16, day 17, day 18, day 19, day 20, or a combination thereof, after the emetogenic therapy.

In some embodiments the method comprises administering to the subject highly long-emetogenic ADC therapy. In some embodiments the method comprises administering to the subject moderately long-emetogenic ADC therapy. In some embodiments the method comprises administering to the subject highly long-emetogenic chemotherapy. In some embodiments the method comprises administering to the subject moderately long-emetogenic chemotherapy.

In some embodiments, the method further comprises administering to the human subject a corticosteroid such as dexamethasone, cortisone, hydrocortisone or prednisone. In a preferred embodiment, the method further comprises administration of dexamethasone according to established anti-emetic guidelines, particularly as described in FDA-approved labels or Herrstedt J. et al, ESMO Open (2024) Vol. 9 Issue 2. Thus, it will be understood that the dexamethasone is administered in a dosage form which is distinct from the netupitant, fosnetupitant, or palonosetron, or salt of the foregoing.

In some embodiments, the method further comprises administering to the human subject olanzapine. In a preferred embodiment, the method further comprises administration of olanzapine according to established anti-emetic guidelines. See Herrstedt J. et al, ESMO Open (2024) Vol. 9 Issue 2.

The antiemetic regimen (step (a) in any of the methods described herein) and the emetogenic therapy (step (b) in any of the methods described herein) can be administered at any time, in any sequence, in any frequency, and at same or different frequencies. However, in a preferred embodiment, the emetogenic therapy is administered after the antiemetic regimen. In another preferred embodiment, the emetogenic therapy is administered no more than 3 hours or 1 hour after the antiemetic regimen. In another preferred embodiment, the emetogenic therapy and antiemetic regimen are administered at the same frequency and duration. I.e., if the emetogenic therapy is administered every twenty days, then the antiemetic regimen is also administered every twenty days. In various embodiments, the administrations of emetogenic therapy and the antiemetic regimen are administered four or more times, eight or more times, 12 or more times, 16 or more times, 20 or more times, 30 or more times, 40 or more times, or 50 or more times.

The antiemetic regimen can be administered in various doses, depending partly on the route of administration. Thus, in one embodiment, when the antiemetic regimen is administered intravenously, the regimen comprises from 100 to 450 mg of fosnetupitant, from 150 to 400 mg of fosnetupitant, or 235 mg of fosnetupitant, wherein the fosnetupitant is optionally administered as a pharmaceutically acceptable salt and the amounts of fosnetupitant are based on the weight of the free base.

In another embodiment, when the antiemetic regimen is administered intravenously, the regimen comprises from 0.1 to 1.0 mg of palonosetron, from 0.2 to 0.8 mg of palonosetron, 0.25 mg of palonosetron, 0.5 mg of palonosetron, or 0.75 mg of palonosetron, wherein the palonosetron is optionally administered as a pharmaceutically acceptable salt and the amounts of palonosetron are based on the weight of the free base.

In another embodiment, when the antiemetic regimen is administered intravenously, the regimen comprises (a) from 100 to 450 mg of fosnetupitant, from 150 to 400 mg of fosnetupitant, or 235 mg of fosnetupitant, wherein the fosnetupitant is optionally administered as a pharmaceutically acceptable salt and the amounts of fosnetupitant are based on the weight of the free base, and (b) from 0.1 to 1.0 mg of palonosetron, from 0.2 to 0.8 mg of palonosetron, 0.25 mg of palonosetron, 0.5 mg of palonosetron, or 0.75 mg of palonosetron, wherein the palonosetron is optionally administered as a pharmaceutically acceptable salt and the amounts of palonosetron are based on the weight of the free base.

A preferred intravenous antiemetic regimen comprises (a) 235 mg of fosnetupitant, optionally administered as a pharmaceutically acceptable salt and (b) 0.25 mg of palonosetron optionally administered as a pharmaceutically acceptable salt. The ingredients (a) and (b) can be administered separately or, preferably, in a single combined form for injection, in association with suitable pharmaceutical excipients and vehicles.

In another embodiment the antiemetic regimen is administered orally and comprises from 100 to 500 mg of netupitant, from 200 to 400 mg of netupitant, or 300 mg of netupitant, wherein the netupitant is optionally administered as a pharmaceutically acceptable salt and the amounts of netupitant are based on the weight of the free base.

In another embodiment the antiemetic regimen is administered orally and comprises from 0.1 to 1.5 mg of palonosetron, from 0.2 to 1.0 mg of palonosetron, 0.25 mg of palonosetron, 0.5 mg of palonosetron, 0.75 mg of palonosetron, or 1.0 mg of palonosetron, wherein the palonosetron is optionally administered as a pharmaceutically acceptable salt and the amounts of palonosetron are based on the weight of the free base.

In another embodiment the antiemetic regimen is administered orally and comprises (a) from 100 to 500 mg of netupitant, from 200 to 400 mg of netupitant, or 300 mg of netupitant, wherein the netupitant is optionally administered as a pharmaceutically acceptable salt and the amounts of netupitant are based on the weight of the free base, and (b) from 0.1 to 1.5 mg of palonosetron, from 0.2 to 1.0 mg of palonosetron, 0.25 mg of palonosetron, 0.5 mg of palonosetron, 0.75 mg of palonosetron, or 1.0 mg of palonosetron, wherein the palonosetron is optionally administered as a pharmaceutically acceptable salt and the amounts of palonosetron are based on the weight of the free base. In this embodiment, a preferred oral antiemetic regimen comprises (a) 300 mg of netupitant, optionally administered as a pharmaceutically acceptable salt and (b) 0.56 mg of palonosetron optionally administered as a pharmaceutically acceptable salt; the ingredients (a) and (b) can be administered separately or, preferably, in a single combined form, more preferably in form of oral suspension, in association with suitable pharmaceutical excipients and vehicles.

In some embodiments the ADC therapy or monoclonal antibody therapy can be characterized by duration of its activity in vivo. Thus, in some embodiments the cycle of ADC therapy comprises a drug released into systemic circulation in nausea and/or vomiting-inducing amounts over a period of 6 or more days, 7 or more days, 8 or more days, 9 or more days, 10 or more days, 11 or more days, 12 or more days, 13 or more days, 14 or more days, 15 or more days, 16 or more days, 17 or more days, 18 or more days, 19 or more days, or 20 or more days after the ADC administration.

In some embodiments the cycle of ADC therapy or monoclonal antibody therapy is defined by a single administration of the ADC or monoclonal antibody over a period of from one week to three or four months, preferably a period of about 1, 2, 3, or 4 weeks, most preferably a period of about 3 weeks or 21 days.

Preferred antibody drug conjugates for practicing the methods of the current disclosure include gemtuzumab ozogamicin, brentuximab vedotin, trastuzumab emtansine, inotuzumab ozogamicin, polatuzumab vedotin, enfortumab vedotin, trastuzumab deruxtecan, sacituzumab govitecan, loncastuximab tesirine, tisotumab vedotin, mirvetuximab soravtansine, datopotamab deruxtecan, luveltamab tazevibulin, patritumab deruxtecan, mecbotamab vedotin, sacituzumab tirumotecan, telisotuzumab vedotin, trastuzumab auristatin, trastuzumab rezetecan, and zilovertamab vedotin; most preferred antibody drug conjugates are trastuxumab deruxtecan and sacituzumab govitecan.

Suitable formulations for palonosetron and netupitant include those currently or previously marketed by Helsinn Healthcare SA (Lugano, Switzerland) and approved by the United States Food and Drug Administration under the brand names Akynzeo® and Aloxi®, in addition to the formulations described in U.S. Pat. No. 10,624,911 B2 to Venturini et al. When administered as combination therapy, the NK1 component can be administered in the same dosage form as the 5HT3 component or in separate dosage forms. However, when dexamethasone or olanzapine is administered, it will be understood that the dexamethasone or olanzapine is preferably administered in a dosage form separate from the NK1 component or 5HT3 component.

In preferred embodiments, the administration of netupitant (or a salt or prodrug thereof, or a salt of the prodrug) optionally in combination with palonosetron (collectively the “netupitant component”) occurs once during any cycle of ADC therapy or chemotherapy or monoclonal antibody therapy. However, it will be understood that the netupitant component can be administered at different frequencies. Thus, for example, the netupitant component can be administered a second time at from 5-10 days after a first administration during a cycle, preferably at 5 days after the first administration, and most preferably at 5 days or 6 days after the first administration during a 21-day cycle of ADC therapy or monoclonal antibody therapy. For clarity, netupitant component (netupitant as single agent or in combination with palonosetron) can be administered on day 1 and on day 5-10 or following days (for example on day 5 or on day 6).

Finally, it will be understood that 5-HT3 antagonists other than palonosetron can be combined with netupitant (or a salt or prodrug thereof, or a salt of the prodrug) in the various methods of the current invention. Alternative 5-HT3 antagonists include, for example, ondansetron, granisetron, dolasetron, and alosetron.

### ALTERNATIVE DESCRIPTION OF EMBODIMENTS

Embodiment 1. A method of preventing ADC-induced nausea and/or vomiting, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

Embodiment 2. A method of achieving a positive no significant nausea outcome following ADC administration, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

Embodiment 3. A method of preventing an ADC-induced side effect selected from fatigue, loss of appetite, and weight loss, preferably fatigue, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

Embodiment 4. A method of preventing ADC-, HEC-, or MEC-induced nausea and/or vomiting, during a long-delayed phase, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

Embodiment 5. A method of achieving a positive no significant nausea outcome following ADC, HEC, or MEC administration, during a long-delayed phase, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

Embodiment 6. A method of preventing an ADC-, HEC-, or MEC-induced side effect selected from fatigue, loss of appetite, and weight loss, preferably fatigue, during a long-delayed phase, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

Embodiment 7. A method of preventing ADC-induced nausea and/or vomiting, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

Embodiment 8. A method of achieving a positive no significant nausea outcome following ADC administration, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

Embodiment 9. A method of preventing an ADC-induced side effect selected from fatigue, loss of appetite, and weight loss, preferably fatigue, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy.

Embodiment 10. A method of preventing ADC-, HEC-, or MEC-induced nausea and/or vomiting in a human subject in need thereof, during a long-delayed phase, to a significantly greater extent than an aprepitant or fosaprepitant regimen, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

Embodiment 11. A method of achieving a positive no significant nausea outcome following ADC, HEC, or MEC administration, during a long-delayed phase, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

Embodiment 12. A method of preventing an ADC-, HEC-, or MEC-induced side effect selected from fatigue, loss of appetite, and weight loss, preferably fatigue, during a long-delayed phase, to a significantly greater extent than an aprepitant or fosaprepitant regimen, in a human subject in need thereof, comprising: (a) administering to the human subject an antiemetic regimen comprising: (i) netupitant or a prodrug thereof, or a pharmaceutically acceptable salt of netupitant or the prodrug; or (ii) palonosetron or a pharmaceutically acceptable salt thereof; or (iii) a combination of (i) and (ii); and (b) administering to the human subject a cycle of moderately or highly emetogenic ADC therapy or chemotherapy.

Embodiment 13. The method of any of embodiments 1-12, wherein the antiemetic regimen comprises (i).

Embodiment 14. The method of any of embodiments 1-12, wherein the antiemetic regimen comprises (ii).

Embodiment 15. The method of any of embodiments 1-12, wherein the antiemetic regimen comprises a combination of (i) and (ii).

Embodiment 16. The method of any of embodiments 1-12, wherein the antiemetic regimen comprises a single administration of the combination of (i) and (ii).

Embodiment 17. The method of any of embodiments 1, 2, 3, 7, 8, and 9, wherein the method achieves the result during a long-delayed phase.

Embodiment 18. The method of any of embodiments 1-6, wherein the method achieves the result to a significantly greater extent than an aprepitant or fosaprepitant regimen.

Embodiment 19. The method of any of embodiments 1, 4, 7, and 10, wherein the prevention of nausea and/or vomiting comprises the prevention of nausea.

Embodiment 20. The method of any of embodiments 1, 4, 7, and 10, wherein the prevention of nausea and/or vomiting comprises the prevention of vomiting.

Embodiment 21. The method of any of embodiments 1, 4, 7, and 10, wherein the prevention of nausea and/or vomiting comprises the prevention of significant nausea.

Embodiment 22. The method of any of embodiments 1, 4, 7, and 10, wherein the prevention of nausea and/or vomiting comprises the prevention of nausea, significant nausea, vomiting, use of rescue medication, or a combination thereof.

Embodiment 23. The method of any of embodiments 1, 4, 7, and 10, wherein the prevention of nausea and/or vomiting comprises complete control, complete response, or complete protection.

Embodiment 24. The method of any of embodiments 1, 4, 7, and 10, wherein the prevention of nausea and/or vomiting comprises an increase in time to treatment failure.

Embodiment 25. The method of any of embodiments 1, 4, 7, and 10, wherein the prevention of nausea and/or vomiting comprises the prevention of nausea and/or vomiting during the acute phase, the delayed phase, the long-delayed phase, the overall phase, or the long-overall phase. Embodiment 26. The method of any of embodiments 1, 4, 7, and 10, wherein the nausea and/or vomiting comprises nausea and/or vomiting through day 7, day 10, day 15, or day 20 after step (b).

Embodiment 27. The method of any of embodiments 1, 4, 7, and 10, wherein the nausea and/or vomiting comprises nausea and/or vomiting during days 6-7, 8-10, 6-10, 11-15, or 16-20, or a combination thereof, after step (b).

Embodiment 28. The method of any of embodiments 1, 4, 7, and 10, wherein the nausea and/or vomiting comprises nausea and/or vomiting experienced on day 6, day 7, day 8, day 9, day 10, day 11, day 12, day 13, day 14, day 15, day 16, day 17, day 18, day 19, day 20, or a combination thereof, after step (b).

Embodiment 29. The method of any of embodiments 3, 6, 9, and 12, wherein the side effect comprises fatigue.

Embodiment 30. The method of any of embodiments 3, 6, 9, and 12, wherein the side effect comprises loss of appetite.

Embodiment 31. The method of any of embodiments 3, 6, 9, and 12, wherein the side effect comprises weight loss.

Embodiment 32. The method of any of embodiments 1-12, comprising administering to the subject highly long-emetogenic ADC therapy.

Embodiment 33. The method of any of embodiments 1-12, comprising administering to the subject moderately long-emetogenic ADC therapy.

Embodiment 34. The method of any of embodiments 4, 5, 6, 10, 11, and 12, comprising administering to the subject highly long-emetogenic chemotherapy.

Embodiment 35. The method of any of embodiments 4, 5, 6, 10, 11, and 12, comprising administering to the subject moderately long-emetogenic chemotherapy.

Embodiment 36. The method of any of the foregoing embodiments, further comprising administering to the human subject a corticosteroid such as dexamethasone, cortisone, hydrocortisone or prednisone.

Embodiment 37. The method of any of the foregoing embodiments, further comprising administering to the human olanzapine.

Embodiment 38. The method of any of the foregoing embodiments, wherein step (b) is performed after step (a).

Embodiment 39. The method of any of the foregoing embodiments, wherein step (b) is performed less than three hours after step (a).

Embodiment 40. The method of any of the foregoing embodiments, wherein (a) and (b) are administered at the same frequency and duration.

Embodiment 41. The method of any of the foregoing embodiments, comprising repeating steps (a) and (b) four or more times, eight or more times, 12 or more times, 16 or more times, 20 or more times, 30 or more times, 40 or more times, or 50 or more times.

Embodiment 42. The method of any of embodiments 1-41, wherein the antiemetic regimen is administered intravenously and comprises: (a) from 100 to 450 mg of fosnetupitant, from 150 to 400 mg of fosnetupitant, or 235 mg of fosnetupitant, wherein the fosnetupitant is optionally administered as a pharmaceutically acceptable salt and the amounts of fosnetupitant are based on the weight of the free base; (b) from 0.1 to 1.0 mg of palonosetron, from 0.2 to 0.8 mg of palonosetron, 0.25 mg of palonosetron, 0.5 mg of palonosetron, or 0.75 mg of palonosetron, wherein the palonosetron is optionally administered as a pharmaceutically acceptable salt and the amounts of palonosetron are based on the weight of the free base; or (c) a combination thereof.

Embodiment 43. The method of any of embodiments 1-41, wherein the antiemetic regimen is administered orally and comprises: (a) from 100 to 500 mg of netupitant, from 200 to 400 mg of netupitant, or 300 mg of netupitant, wherein the netupitant is optionally administered as a pharmaceutically acceptable salt and the amounts of netupitant are based on the weight of the free base; (b) from 0.1 to 1.5 mg of palonosetron, from 0.2 to 1.0 mg of palonosetron, 0.25 mg of palonosetron, 0.5 mg of palonosetron, 0.75 mg of palonosetron, or 1.0 mg of palonosetron, wherein the palonosetron is optionally administered as a pharmaceutically acceptable salt and the amounts of palonosetron are based on the weight of the free base; or (c) a combination thereof.

Embodiment 44. The method of any of embodiments 1-41, wherein the antiemetic regimen is administered intravenously and comprises: (a) from 100 to 450 mg of fosnetupitant, from 150 to 400 mg of fosnetupitant, or 235 mg of fosnetupitant, wherein the fosnetupitant is optionally administered as a pharmaceutically acceptable salt and the amounts of fosnetupitant are based on the weight of the free base; and (b) from 0.1 to 1.0 mg of palonosetron, from 0.2 to 0.8 mg of palonosetron, 0.25 mg of palonosetron, 0.5 mg of palonosetron, or 0.75 mg of palonosetron, wherein the palonosetron is optionally administered as a pharmaceutically acceptable salt and the amounts of palonosetron are based on the weight of the free base.

Embodiment 45. The method of any of embodiments 1-41, wherein the antiemetic regimen is administered orally and comprises: (a) from 100 to 500 mg of netupitant, from 200 to 400 mg of netupitant, or 300 mg of netupitant, wherein the netupitant is optionally administered as a pharmaceutically acceptable salt and the amounts of netupitant are based on the weight of the free base; and (b) from 0.1 to 1.5 mg of palonosetron, from 0.2 to 1.0 mg of palonosetron, 0.25 mg of palonosetron, 0.5 mg of palonosetron, 0.75 mg of palonosetron, or 1.0 mg of palonosetron, wherein the palonosetron is optionally administered as a pharmaceutically acceptable salt and the amounts of palonosetron are based on the weight of the free base.

Embodiment 46. The method of any of embodiments 1-41, wherein the antiemetic regimen is administered intravenously and comprises: (a) 235 mg of fosnetupitant or a pharmaceutically acceptable salt thereof, wherein the amount of fosnetupitant is based on the weight of the free base; and (b) 0.25 mg of palonosetron or a pharmaceutically acceptable salt thereof, wherein the amount of palonosetron is based on the weight of the free base.

Embodiment 47. The method of any of embodiments 1-41, wherein the antiemetic regimen is administered orally and comprises: (a) 300 mg of netupitant or a pharmaceutically acceptable salt thereof, wherein the amount of netupitant is based on the weight of the free base; and (b) 0.5 mg of palonosetron or a pharmaceutically acceptable salt and the amount of palonosetron is based on the weight of the free base.

Embodiment 48. The method of any of the foregoing embodiments wherein the antiemetic regimen comprises a single administration of netupitant or a prodrug thereof, or a pharmaceutically acceptable salt of netupitant or the prodrug; and a single administration of palonosetron or a pharmaceutically acceptable salt thereof, per cycle of the emetogenic therapy.

Embodiment 49. The method of any of the foregoing embodiments, wherein the treatment effect is observed during a time period selected from hours 120-168, 120-216, 120-264, 120-312, 120-360, 120-408, 120-456, 120-480, 168-216, 168-264, 168-312, 169-360, 168-408, 168-456, 168-480, 216-264, 216-312, 216-360, 216-408, 216-456, 216-480, 264-312, 264-360, 264-408, 264-456, or 264-480, 312-360, 312-408, 312-456, or 312-480, 360-408, 360-456, 360-480, 408-456, 408-480, 456-480, 120-240, 120-360, 120-480, 240-360, 240-480, and 360-480, after the administration of the highly emetogenic ADC therapy, the moderately emetogenic ADC therapy, the highly emetogenic chemotherapy, or the moderately emetogenic chemotherapy.

Embodiment 50. The method of any of embodiments 1-49, wherein the treatment effect is observed from hours 120-240, after the administration of the highly emetogenic ADC therapy, the moderately emetogenic ADC therapy, the highly emetogenic chemotherapy, or the moderately emetogenic chemotherapy.

Embodiment 51. The method of any embodiments 1-49, wherein the treatment effect is observed from hours 120-360, after the administration of the highly emetogenic ADC therapy, the moderately emetogenic ADC therapy, the highly emetogenic chemotherapy, or the moderately emetogenic chemotherapy.

Embodiment 52. The method of any of embodiments 1-49, wherein the treatment effect is observed from hours 120-480, after the administration of the highly emetogenic ADC therapy, the moderately emetogenic ADC therapy, the highly emetogenic chemotherapy, or the moderately emetogenic chemotherapy.

Embodiment 53. The method of any of the foregoing embodiments, wherein the cycle of ADC therapy comprises a drug released into systemic circulation in nausea and/or vomiting-inducing inducing amounts over a period of 6 or more days, 7 or more days, 8 or more days, 9 or more days, 10 or more days, 11 or more days, 12 or more days, 13 or more days, 14 or more days, 15 or more days, 16 or more days, 17 or more days, 18 or more days, 19 or more days, or 20 or more days after the ADC administration.

Embodiment 54. The method of any of the foregoing embodiments, wherein the monoclonal antibody drug conjugate is selected from gemtuzumab ozogamicin, brentuximab vedotin, trastuzumab emtansine, inotuzumab ozogamicin, polatuzumab vedotin, enfortumab vedotin, trastuzumab deruxtecan, sacituzumab govitecan, loncastuximab tesirine, tisotumab vedotin, mirvetuximab soravtansine, datopotamab deruxtecan, luveltamab tazevibulin, patritumab deruxtecan, mecbotamab vedotin, sacituzumab tirumotecan, telisotuzumab vedotin, trastuzumab auristatin, trastuzumab rezetecan, and zilovertamab vedotin.

Embodiment 55. The method of any of the foregoing embodiments, wherein the monoclonal antibody drug conjugate is selected from sacituzumab govitecan and trastuzumab deruxtecan.

Embodiment 56. The method of any of the foregoing embodiments, wherein netupitant or a prodrug thereof, or a pharmaceutically acceptable salt of netupitant or the prodrug, is administered.

Embodiment 57. The method of any of the foregoing embodiments, wherein netupitant or a prodrug thereof, or a pharmaceutically acceptable salt of netupitant or the prodrug, is administered, in combination with a 5-HT3 antagonist, preferably palonosetron or a pharmaceutically acceptable salt thereof.

### EXAMPLES

### Example 1: NK1 Receptor Occupancy by Netupitant During the Long-Delayed Phase

Data from previous pharmacokinetic (PK) studies in a Caucasian population were described by PK models and the rate, extent, and duration of NK₁ receptor occupancy (RO) with netupitant were predicted by pharmacodynamic modeling in their respective relevant tissues (Baron-Hay, Supp Care Cancer 2019). This model was applied in this analysis to determine the netupitant NK₁ RO percent in the striatum region up to 480 h, for netupitant and aprepitant.

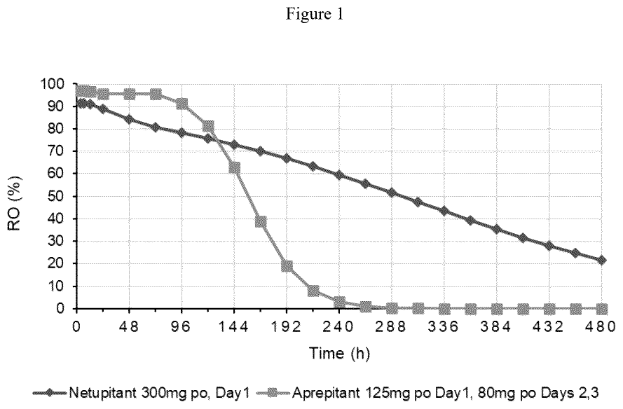

Netupitant NK₁ RO was predicted to reach 90% at 2.2 h after netupitant administration, based on an oral dose of 300 mg of netupitant or a 235 mg intravenous dose of fosnetupitant (both doses based on weight of free base). RO began to decline slowly after 24 h, reaching 70% at 168 h (Day 7) 60% at 240 h (Day 10), and 20% at 480 h (day 20), as reported in FIG. 1.

For a standard regimen of aprepitant (125 mg aprepitant iv on day 1 followed by 80 mg po on days 2 and 3) NK₁ RO was predicted to remain above 90% for 96 hours, but thereafter to rapidly decline, falling to 60% RO after 144 hours and almost zero after 240 hours.

### Example 2: Protocol for Evaluating the Efficacy of a Combination of Netupitant and Palonosetron Against ADC-Induced Nausea and Vomiting

This study will be an open label, active controlled, multicenter, randomized phase 3 study. Eligible patients will be randomized to receive either netupitant (as netupitant or fosnetupitant), palonosetron, and dexamethasone (the NEPA arm) or aprepitant (as aprepitant or fosaprepitant), a standard of care 5HT3 antagonist, and dexamethasone (the APR arm). Both regimens will be administered according to antiemetic guidelines established for highly emetogenic chemotherapy. (Herrstedt J. et al, ESMO Open (2024) Vol. 9 Issue 2).

Patients with cancer scheduled to receive trastuzumab deruxtecan and potentially other long-delayed chemotherapy agents (stratified according to treatment) for the first time will be enrolled in this study. Patients will be allowed to take rescue medication for CINV, if necessary, throughout the study period. Data will be collected from patient diaries in which patients report daily the incidence of vomiting or use of rescue medication. In addition, patients will report the presence of nausea, fatigue, and decreased appetite using a VAS scale or a four-item scale rating the severity as none, mild, moderate, and severe. Data will be collected daily for up to four 21-day cycles of ADC therapy.

Effects to be measured and analyzed include:
Daily rates of various endpoints, including nausea, nausea severity, vomiting, CR, CC, and CP.
CR rates during the acute, delayed, long-delayed, overall, and long-overall phases (applying various definitions for the long-delayed phase).
CC rates during the acute, delayed, long-delayed, overall, and long-overall phases (applying various definitions for the long-delayed phase).
CP rates during the acute, delayed, long-delayed, overall, and long-overall phases (applying various definitions for the long-delayed phase).
Time to treatment failure.
Nausea severity.
Effects on appetite and weight loss.

Data will be analyzed and compared against other studies to determine:
Superiority of the netupitant/palonosetron regimen to existing aprepitant-based antiemetic drug regimens.
The independent effect of palonosetron against nausea and/or vomiting in the long-delayed phase.

### Example 3: Efficacy of a Combination of Netupitant and Palonosetron Against ADC-Induced Nausea and Vomiting in Patients with Advanced Breast Cancer (ABC) Treated with Trastuzumab/Deruxtecan

This analysis investigated the impact of NEPA on nausea and vomiting in the first cycle on maintaining T-DXd dosing over time.

Methods: Data from patients with HER2-positive ABC treated with at least two cycles of T-DXd were extracted and analyzed from the clinical data warehouse of Samsung Medical Center. Patients were grouped based on the use of a guideline-recommended triple antiemetic regimen (TAR) in the first cycle of T-DXd. The TAR consisted of either the fixed combination of netupitant/palonosetron (NEPA) or the combination of an NK1 receptor antagonist (RA) and a 5-HT3 RA plus dexamethasone. The proportions of patients requiring a reduction from the 5.4 mg/kg starting dose of T-DXd to 4.4 mg/kg and 3.2 mg/kg were calculated for both TAR and non-TAR groups.

Results: 59 patients with HER2-positive ABC received a median of 7 cycles (range, 2-30) of T-DXd. The median age at initiation of T-DXd was 55 (range, 28-79). Prophylactic TAR was administered to 49 (83%) patients in the first T-DXd cycle: 46 with NEPA and 3 with other NK1 RA and 5-HT3 RA combinations. Ten patients (17%) initially received either a 5-HT3 RA alone (n=9) or no prophylaxis (n=1). Patients who received TAR in cycle 1 were less likely to require T-DXd dose reduction to ≤4.4 mg/kg (24% vs 50%, p=0.133) or ≤3.2 mg/kg (0% vs 10%, p=0.169), with a significantly greater magnitude of dose reduction observed in patients who did not receive TAR in cycle 1 than those who did (P=0.011). In the course of the study, 8 of 9 patients initially on a 5-HT3 RA alone and 2 of 3 patients initially on other NK1 RA and 5-HT3 RA combinations switched to NEPA, due to the high incidence of nausea and vomiting. At the data cutoff, no patients discontinued T-DXd due to intractable nausea and vomiting; 44 (75%) patients continued to receive T-DXd, while 8 (14%), 4 (7%), and 2 (3%) patients discontinued due to disease progression, death, or pneumonia/pneumonitis, respectively. One patient was lost to follow-up after cycle 2.

Conclusions: These results highlight the previously unreported finding that administering guideline-recommended TAR prophylaxis facilitates maintaining the dose intensity of T-DXd throughout treatment. Using the present upfront antiemetic regimen when initiating T-DXd is critical to optimize patient outcomes.

### Example 4: Analysis of Nausea and Vomiting Probability over Multiple Cycles of NEPA (Netupitant/Palonosetron) Prophylaxis on Metastatic Breast Cancer Patients Treated with Trastuzumab Deruxtecan

This retrospective analysis examined the probability of patients experiencing NV over multiple cycles of T-DXd following antiemetic prophylaxis with NEPA, a fixed-combination of an NK1 RA (netupitant/fosnetupitant) and 5-HT3 RA (palonosetron).

Methods: Occurrence and severity of NV in patients with HER2-positive or HER2-low MBC over the first 5 cycles of T-DXd for each patient were extracted and analyzed from clinical records at a single-center.

Probability of NV occurring in each of 5 cycles of T-DXd was estimated using a logistic regression with NV as a dependent variable, cycle as an independent variable and with Generalized Estimating Equation to account for repeated measures.

Results: Twenty-five patients were included for a total of 118 cycles. NEPA with dexamethasone was administered as prophylaxis in all but 6 cycles. The median age at T-DXd initiation was 64 years (range, 41-78). The estimated probability of developing NV was low and diminished over each cycle (Table 1), suggesting that NV was well-controlled with NEPA from the first cycle onward.

TABLE 1
Probability of NV Occurrence During Each Cycle
(Estimates Obtained from a Logistic Regression.

| | Chances of NV | | |
|---|---|---|---|
| Cycle | occurrence | Lower 95% CI | Upper 95% CI |
| 1 | 0.2800 | 0.1397 | 0.4822 |
| 2 | 0.1200 | 0.0392 | 0.3130 |
| 3 | 0.0812 | 0.0192 | 0.2856 |
| 4 | 0.0390 | 0.0042 | 0.2812 |
| 5 | 0.0408 | 0.0026 | 0.4101 |

Conclusion: These findings indicate that NEPA is likely to be highly effective in preventing NV over multiple cycles in MBC patients receiving T-DXd. It also underscores the value of using an appropriate upfront antiemetic regimen from the first cycle of T-DXd.

### Example 5: Analysis of the Association Between Nausea, Vomiting, and Fatigue in Metastatic Breast Cancer Patients Treated with Trastuzumab Deruxtecan

This analysis examined the association between N&V in one cycle and fatigue in the next.

Methods: Occurrence and severity of N&V and fatigue in patients with HER2+/HER2-low MBC over 5 cycles of T-DXd were extracted/analyzed from clinical records at a single-center. Association between occurrence of Fatigue and previous N&V was tested using a logistic regression with Generalized Estimating Equation and only mild adjustment to account repeated measures.

Results: Twenty-five patients were included for a total of 118 cycles. All but 1 patient received antiemetic prophylaxis from first cycle; most received NEPA, a fixed-combination of an NK1RA (netupitant/fosnetupitant) and 5-HT3RA (palonosetron), with dexamethasone. The median age at T-DXd initiation was 64 years (range, 41-78). N&V was well-controlled with NEPA. While not statistically significant, the odds ratio suggests that patients with prior any Grade NV may have a higher likelihood of experiencing fatigue compared to those without prior NV (Table 2). Consistently, the estimated probability of developing fatigue was higher in patients who experienced NV in the prior cycle (45.8% vs. 21.8%, Table 3).

TABLE 2
Results of a Logistic Regression Analysis Testing the
Association Between Occurrence of Fatigue and Prior NV.

| | Odds | Lower | Upper | |
|---|---|---|---|---|
| Parameter | Ratio | 95% CI | 95% CI | P-value |
| Previous CINV | 3.038 | 0.497 | 18.573 | 0.2290 |
| (yes vs. no) | | | | |
| Treatment Cycle 2 vs. 1 | 1.365 | 0.481 | 3.872 | 0.5588 |
| Treatment Cycle 3 vs. 1 | 0.895 | 0.280 | 2.861 | 0.8510 |
| Treatment Cycle 4 vs. 1 | 1.988 | 0.526 | 7.505 | 0.3109 |
| Treatment Cycle 5 vs. 1 | 1.409 | 0.317 | 6.262 | 0.6524 |

TABLE 3
Probability of Developing Fatigue as a Function of the
Presence or Absence of NV in the Prior Cycle. Estimates
Obtained from the Logistic Model of Table 2.

| | Probability to | Lower | Upper |
|---|---|---|---|
| Previous CINV | Develop Fatigue | 95% CI | 95% CI |
| YES | 0.4579 | 0.1370 | 0.8181 |
| NO | 0.2176 | 0.1025 | 0.4037 [symbol] |

Conclusion: These findings suggest a potential link between prior NV and fatigue, indicating that controlling NV may help manage fatigue in MBC patients receiving T-DXd. More data are needed to confirm.

### Example 5: Effectiveness of Intravenous Fosnetupitant & Palonosetron for CINV Prophylaxis in Patients Receiving Highly Emetogenic Chemotherapy Regimens

A phase IV, multicentric, open label, single arm prospective study was conducted at six centers following individual Institutional Ethics Committee (CTRI/2023/04/051951). Complete response (CR) (no vomiting and no rescue medication), complete protection (CP) [CR+no significant nausea (<25 mm) on visual analogue scale], complete control (CC) [CR+no nausea (<5 mm)] were evaluated during acute (0-24 hrs), delayed (>24-120 hrs) and extended delayed (>120-240 hrs) phases. Safety was evaluated up to 240 hrs.

Results: Out of total 178 patients 90 (50.56%) patients received HEC regimen with Cisplatin-Paclitaxel (19.10%) being the most common regimen. CR in acute, delayed and extended delayed phases were 74.44%, 90.00% and 94.44%, respectively. Furthermore, CP and CC were 73.33% and 47.78%, 87.78% and 71.11% and 94.44% and 84.44% in acute, delayed and extended delayed phases respectively (Table 1). Overall, the intravenous administration of NEPA was generally well tolerated, with only 17 (9.55%) patients experiencing adverse effects. The most common adverse effects reported were headache in 4 (2.25%) patients and injection site reactions in 3 (1.68%) patients. One serious adverse event, unrelated to the study drug was reported. The efficacy results are presented in Table 4.

TABLE 4
CINV Efficacy Assessment in patients receiving HEC regimen.

| | | | Extended | |
|---|---|---|---|---|
| | Acute Phase | Delayed phase | Delayed phase | Overall phase |
| PARAMETER | (0-24 hrs) | (>24-120 hrs) | (>120-240 hrs) | (0-120 hrs) |
| Complete Response | 67 (74.44%) | 81 (90.00%) | 85 (94.44%) | 65 (72.22%) |
| (CR) | | | | |
| Complete Protection | 66 (73.33%) | 79 (87.78%) | 85 (94.44%) | 63 (70.00%) |
| (CP) | | | | |
| Complete Control | 43 (47.78%) | 64 (71.11%) | 76 (84.44%) | 40 (44.44%) |
| (CC) | | | | |

Conclusion: IV NEPA demonstrated high efficacy and excellent tolerability in a real world setting, exhibiting complete response rates of ≥90% in both delayed and extended delayed phases in patients receiving HEC regimens.

The present data support for NEPA a carry-over effect, i.e. maintaining over time the optimal response achieved in the first days after administration, alongside with the efficacy provided by higher/longer receptor occupancy. This is very important because, as well-known, once triggered, the chemotherapeutic-induced emetic or nausea stimulus is difficult to control: the carry over effect is therefore an issue in case of sub-optimal control of CINV.

From the foregoing it will be appreciated that, although specific embodiments of the disclosure have been described herein for purposes of illustration, various modifications may be made without deviating from the spirit and scope of the disclosure.

1. An antiemetic regimen comprising;
a. administering to a human subject:
i. netupitant or a prodrug thereof, or a pharmaceutically acceptable salt of netupitant or the prodrug; optionally in combination with
ii. palonosetron or a pharmaceutically acceptable salt thereof; and
b. administering to the human subject a cycle of an emetogenic treatment selected from ADC therapy, chemotherapy, or monoclonal antibody therapy,
for use in a method of preventing nausea and/or vomiting induced by the emetogenic treatment during a long-delayed phase, in a human subject in need thereof.

2. An antiemetic regimen comprising;
a. administering to a human subject:
i. netupitant or a prodrug thereof, or a pharmaceutically acceptable salt of netupitant or the prodrug; optionally in combination with
ii. palonosetron or a pharmaceutically acceptable salt thereof; and
b. administering to the human subject a cycle of a emetogenic treatment selected from ADC therapy and chemotherapy,
for use in a method of preventing nausea and/or vomiting induced by the emetogenic treatment during a long-delayed phase, in a human subject in need thereof.

3. An antiemetic regimen comprising;
a. administering to a human subject:
i. netupitant or a prodrug thereof, or a pharmaceutically acceptable salt of netupitant or the prodrug; optionally in combination with
ii. palonosetron or a pharmaceutically acceptable salt thereof; and
b. administering to the human subject a cycle of an emetogenic treatment selected from ADC therapy and monoclonal antibody therapy,
for use in a method of preventing nausea and/or vomiting induced by the emetogenic treatment, in a human subject in need thereof.

4. An antiemetic regimen comprising;
a. administering to a human subject:
i. netupitant or a prodrug thereof, or a pharmaceutically acceptable salt of netupitant or the prodrug; optionally in combination with
ii. palonosetron or a pharmaceutically acceptable salt thereof; and
b. administering to the human subject a cycle of an emetogenic treatment comprising ADC therapy,
for use in a method of preventing nausea and/or vomiting induced by the emetogenic treatment, in a human subject in need thereof.

5. The regimen of claim 3 or 4, for use in a method of preventing nausea and/or vomiting induced by the emetogenic therapy during a long-delayed phase.

6. The regimen of any of claims 1-4, for achieving a positive no significant nausea outcome following said emetogenic treatment, during a long-delayed phase.

7. The regimen of any of claims 1-4, wherein the prevention of nausea and/or vomiting comprises preventing the use of rescue medication following said emetogenic treatment.

8. The regimen of any of claims 1-4, wherein the prevention of nausea and vomiting comprises achieving complete control, complete response, or complete protection.

9. The regimen of any of claims 1-4 wherein the prevention of nausea and/or vomiting comprises an increase in time to treatment failure.

10. The regimen of any of claims 1-4, for preventing a side effect induced by the emetogenic treatment selected from fatigue, loss of appetite, and weight loss, during a long-delayed phase.

11. The regimen of any of claims 1-4, for preventing fatigue induced by said emetogenic treatment.

12. The regimen of any of claims 1-4, for preventing loss of appetite induced by said emetogenic treatment.

13. The regimen of any of claims 1-4, for preventing weight loss induced by said emetogenic treatment.

14. The regimen of claims any of claims 1-4, wherein one or more of: preventing nausea and/or vomiting during a long-delayed phase, achieving a positive no significant nausea outcome during a long-delayed phase, or preventing a side effect selected from fatigue, loss of appetite, and weight loss, during a long-delayed phase, occurs to a significantly greater extent than an aprepitant or fosaprepitant regimen.

15. The regimen of any of claims 1-14, wherein the antiemetic regimen comprises a combination of (i) and (ii).

16. The regimen of any of claims 1-14, wherein the antiemetic regimen comprises a single administration of the combination of (i) and (ii) during the cycle.

17. The regimen of any of claims 1-16, wherein the prevention of nausea and/or vomiting comprises the prevention of nausea and/or vomiting during the acute phase, the delayed phase, the long-delayed phase, the overall phase, and/or the long-overall phase.

18. The regimen of any of claims 1-17, wherein, when nausea and/or vomiting it prevented during the long delayed phase, the nausea and/or vomiting comprises nausea and/or vomiting through day 7, day 10, day 15, or day 20 after step (b).

19. The regimen of any of claims 1-17, wherein, when nausea and/or vomiting it prevented during the long delayed phase, the nausea and/or vomiting comprises nausea and/or vomiting during days 6-7, 8-10, 6-10, 11-15, or 16-20, or a combination thereof, after step (b).

20. The regimen of any of claims 1-17, wherein, when nausea and/or vomiting it prevented during the long delayed phase, the nausea and/or vomiting comprises nausea and/or vomiting experienced on day 6, day 7, day 8, day 9, day 10, day 11, day 12, day 13, day 14, day 15, day 16, day 17, day 18, day 19, day 20, or a combination thereof, after step (b).

21. The regimen of any of claims 1-20, wherein the emetogenic treatment is highly emetogenic.

22. The regimen of any of claims 1-20, wherein the emetogenic treatment is moderately emetogenic.

23. The regimen of any of claims 1-20, wherein, when chemotherapy is administered, administering HEC or MEC.

24. The regimen of any of claims 1-20 comprising, when ADC therapy is administered, administering to the subject highly long-emetogenic ADC therapy.

25. The regimen of any of claims 1-20 comprising, when ADC therapy is administered, administering to the subject moderately long-emetogenic ADC therapy.

26. The regimen of any of the foregoing claims, further comprising administering to the human subject a corticosteroid such as dexamethasone, cortisone, hydrocortisone or prednisone.

27. The regimen of any of the foregoing claims, further comprising administering to the human olanzapine.

28. The regimen of any of the foregoing claims, wherein step (b) is performed after step (a).

29. The regimen of any of the foregoing claims, wherein step (b) is performed less than three hours after step (a).

30. The regimen of any of the foregoing claims, wherein (a) and (b) are administered at the same frequency and duration.

31. The regimen of any of the foregoing claims, wherein steps (a) and (b) are performed sequentially, simultaneously, or in any order.

32. The regimen of any of the foregoing claims, wherein the cycle in step (b) is performed as a single administration over a period of from one week to four months, preferably of about 21 days.

33. The regimen of any of the foregoing claims, wherein the cycle in step (b) is performed as a single administration over a period of from one week to four months, preferably of about 21 days, and the netupitant or a prodrug thereof, or pharmaceutically acceptable salt of netupitant or the prodrug, is administered once during the cycle.

34. The regimen of any of the foregoing claims, wherein the cycle in step (b) is performed as a single administration over a period of from one week to four months, preferably of about 21 days, and the netupitant or a prodrug thereof, or pharmaceutically acceptable salt of netupitant or the prodrug, is administered twice during the cycle, preferably approximately 5 days apart.

35. The regimen of any of the foregoing claims, comprising repeating steps (a) and (b) four or more times, eight or more times, 12 or more times, 16 or more times, 20 or more times, 30 or more times, 40 or more times, or 50 or more times.

36. The regimen of any of claims 1-35, step (a) being administered intravenously and comprising:
a. from 100 to 450 mg of fosnetupitant, from 150 to 400 mg of fosnetupitant, or 235 mg of fosnetupitant, wherein the fosnetupitant is optionally administered as a pharmaceutically acceptable salt and the amounts of fosnetupitant are based on the weight of the free base;
b. from 0.1 to 1.0 mg of palonosetron, from 0.2 to 0.8 mg of palonosetron, 0.25 mg of palonosetron, 0.5 mg of palonosetron, or 0.75 mg of palonosetron, wherein the palonosetron is optionally administered as a pharmaceutically acceptable salt and the amounts of palonosetron are based on the weight of the free base; or
c. a combination thereof.

37. The regimen of any of claims 1-35, step (a) being administered orally and comprising:
a. from 100 to 500 mg of netupitant, from 200 to 400 mg of netupitant, or 300 mg of netupitant, wherein the netupitant is optionally administered as a pharmaceutically acceptable salt and the amounts of netupitant are based on the weight of the free base;
b. from 0.1 to 1.5 mg of palonosetron, from 0.2 to 1.0 mg of palonosetron, 0.25 mg of palonosetron, 0.5 mg of palonosetron, 0.75 mg of palonosetron, or 1.0 mg of palonosetron, wherein the palonosetron is optionally administered as a pharmaceutically acceptable salt and the amounts of palonosetron are based on the weight of the free base; or
c. a combination thereof.

38. The regimen of any of the foregoing claims, wherein when ADC therapy is administered, the cycle of ADC therapy comprises a drug released into systemic circulation in nausea and/or vomiting-inducing amounts over a period of 6 or more days, 7 or more days, 8 or more days, 9 or more days, 10 or more days, 11 or more days, 12 or more days, 13 or more days, 14 or more days, 15 or more days, 16 or more days, 17 or more days, 18 or more days, 19 or more days, or 20 or more days after the ADC administration.

39. The regimen of any of the foregoing claims wherein, when ADC therapy is administered, the ADC is selected from gemtuzumab ozogamicin, brentuximab vedotin, trastuzumab emtansine, inotuzumab ozogamicin, polatuzumab vedotin, enfortumab vedotin, trastuzumab deruxtecan, sacituzumab govitecan, loncastuximab tesirine, tisotumab vedotin, mirvetuximab soravtansine, datopotamab deruxtecan, luveltamab tazevibulin, patritumab deruxtecan, mecbotamab vedotin, sacituzumab tirumotecan, telisotuzumab vedotin, trastuzumab auristatin, trastuzumab rezetecan, and zilovertamab vedotin.

40. The regimen of any of the foregoing claims wherein, when ADC therapy is administered, the antibody drug conjugate is selected from sacituzumab govitecan and trastuzumab deruxtecan.

41. The regimen of any of the foregoing claims, wherein netupitant or a prodrug thereof, or a pharmaceutically acceptable salt of netupitant or the prodrug, is administered in combination with a 5-HT3 antagonist.

42. The regimen of any of claims 1-4 wherein the method prevents nausea and/or vomiting induced by the emetogenic treatment during a long-delayed phase of from 120 to 240 hours.

43. The regimen of any of the foregoing claims wherein the method prevents nausea and/or vomiting induced by the emetogenic treatment during a long-delayed phase of from 120 to 240 hours.

44. The regimen of any of claims 1-4 wherein the method prevents nausea and/or vomiting induced by the emetogenic treatment during a long-delayed phase of from 120 to 480 hours.

45. The regimen of any of the foregoing claims wherein the method prevents nausea and/or vomiting induced by the emetogenic treatment during a long-delayed phase of from 120 to 480 hours.
